(19) United States
(12) Patent Application Publication
(10) Pub. No.: US 2017/0035687 A1
(43) Pub. Date: Feb. 9, 2017

(54) Skin treatment formulations

(71) Applicants: Sanjay L. Dhar, Irvine, CA (US); Mohammad Ali Kharazmi, Corona Del Mar, CA (US); Shahorkh Zekavati, Aliso Viejo, CA (US)

(72) Inventors: Sanjay L. Dhar, Irvine, CA (US); Mohammad Ali Kharazmi, Corona Del Mar, CA (US); Shahorkh Zekavati, Aliso Viejo, CA (US)

(21) Appl. No.: 15/305,636

(22) PCT Filed: Apr. 21, 2014
(86) PCT No.: PCT/US14/34738

§ 371 Date: Oct. 20, 2016

(51) Int. Cl. A61K 8/98; A61K 8/92; A61Q 19/00; A61Q 5/02; A61Q 5/12; A61Q 19/10; A61Q 19/02; A61Q 7/00

(52) U.S. Cl. CPC A61K 8/981; A61Q 19/02; A61K 8/922; A61Q 7/00; A61Q 5/02; A61Q 5/12; A61Q 19/10; A61Q 19/00; A61K 2800/80

(57) ABSTRACT

Dermal treatment compositions include medium recovered from an adipose-derived stem cell culture where the medium is collected under defined conditions. The medium includes growth factors and other materials that, when transported to living portions of the skin, improve the condition of damaged or aged skin. The compositions include combinations of transport ingredients in controlled proportions for effective topical application.

### BACKGROUND OF THE INVENTION

Technical Field

This invention is in the field of dermal care and treatment. In particular, it concerns compositions that improve the condition of damaged skin by topical application.

Background

Skin may be damaged by a variety of insults including aging, sun exposure, wound healing, and cosmetic treatments. In particular, as skin ages, circulation lessens and dermal fibroblast cells become less active; skin becomes thinner and loses elasticity. Aged skin shows a decrease in volume and elasticity. There is a need to prevent and reverse these conditions to maintain healthy, youthful skin.

It is very difficult to repair such damage and address such loss of function by topically applied agents because the skin includes an external layer, the stratum corneum, that is largely impervious to most aqueous materials. The layer is formed when living keratinocytes are transformed into non-living corneocytes. In these surface cells, the cell membrane is replaced by a layer of ceramides covalently linked to and enveloping structural proteins. Corneal cells include a dense protein network of keratin. This protein helps maintain hydration by preventing water loss. This barrier also reduces the ability of topical medicaments to reach the living layers of the skin.

Various physical methods are available to introduce materials to living tissue through the stratum corneum. These include dermabrasion, chemical peels, carrier solvents such as DMSO, and electroporation. Each of these produces discomfort and may cause further skin damage.

Many tissues, including living layers of the skin, respond to appropriate mixtures of growth factors to encourage regeneration. This may be useful in direct application to exposed living tissue as in treated bandages and the like. U.S. Pat. No. 7,118,746 to Naughton et al. describes materials derived from three-dimensional cultures of diverse cell types used for wound and burn healing. US 2013/0110132 to Epstein et al. discloses compositions including processed conditioned medium from cultured stem cells purported to facilitate healing of injured tissue, including burned skin, broken bone, torn tendons, and decubitus ulcers.

However, as discussed above, the relatively impervious nature of the stratum corneum blocks most topical applications from reaching living layers of the skin. There is thus a need for effective medicaments that can treat damaged skin without direct exposure to the living tissue.

US 2012/0195969 to Riordan etal. describes administration of compositions including processed conditioned medium from cultured stem cells applied topically to treat acne. Acne is associated with obstruction of sebaceous follicles that communicate with the outer surface of the skin. Thus, topical application may be effective for acne in some cases without the need to penetrate to the living layers of the skin other than in the sebaceous follicles.

Cultured cells may include infectious agents such as viruses, mycoplasma, bacteria, and fungus. Such infectious agents may, in some cases, be present in the original tissue from which the stem cells arise. Good sterile technique can reduce the likelihood of subsequent contamination but even use of antimicrobials may not destroy preexisting infectious agents. There is thus a risk that infectious agents derived from cultured cells may infect the treatment site of treated patients. There is a need to provide dermal treatments without the risk of infection by tissue-derived infectious agents.

### SUMMARY

In some embodiments, the invention includes dermal treatment compositions comprising a medium recovered from human adipose-derived stem cell culture. The culture may be an adherent culture having from about 40% to about 90% confluence. The compositions may include a mixture of two changes of media removed less than about 60 hours apart.

The dermal treatment composition may include medium from cell culture that reaches a confluence of about 70% between the two changes of media. The time between the two changes of media may be about 48-72 hours, and may be about 48 hours. The culture may be maintained in a flat-bottom vessel having a fill level of medium is about 0.15 to about 0.25 cm. Useful vessels include culture flasks with areas 75, 150, 165, 175, or 225 cm² and fill levels of about 12-50 mL.

Some embodiments include dermal treatment compositions that may be non-rinse compositions including about 40% to about 60% of the medium, about 10% to about 30% of organic phase materials, and about 2% to about 5% of surfactants/emulsifiers by weight.

Other embodiments include one or more fats such as coconut oil, avocado oil, or caprylic/capric triglyceride. Other compositions may include whitening agents.

In other embodiments the invention includes a treatment method for washing the hair with two described dermal treatment compositions. The first composition includes about 10% of the medium and about 70% of surfactants/emulsifiers by weight. The second composition includes about 5% coconut oil, about 75% of stem cell derived medium and about 5% to about 10% of surfactants/emulsifiers by weight. The method includes steps of washing the hair with the first composition, rinsing the hair with water to remove the first composition, applying to the hair the second composition, and rinsing the hair with water to remove the second composition. The second composition may remain in the hair for a longer time than the first composition.

In other embodiments the invention includes dermal treatment compositions for treating the skin of an individual, where the compositions include medium recovered from human adipose-derived stem cell culture. The stem cells used for the culture derive from cells of the individual treated. Such compositions can include a range of other ingredients depending on the targeted portion of the skin.

### DETAILED DESCRIPTION

This invention includes compositions containing stem cell derived growth factors, cytokines, stress proteins, and nutrients (collectively, stem cell products). In addition the compositions contain a combination of carriers and support materials. Without intent to be bound by theory, we believe that the combination of carriers and support materials serve to convey the stem cell products to the living layers of skin. The routes may include direct diffusion through the stratum corneum and entry through one or more of hair follicles, sebaceous glands, and eccrine glands of the skin. Once beyond the stratum corneum, the stem cell products may relatively freely diffuse about the living dermal layers.

In some embodiments, the compositions of our invention may be targeted to particular routes, such as through hair follicles, by selection of appropriate proportions of carriers and support materials. Entry through hair follicles is of particular value for compositions intended to augment or improve hair growth or quality.

In some embodiments, the compositions of our invention include a mixture of active growth factors and cytokines (including TGF-B, PDGF, and GM-CSG, interleukins, and matrix proteins) produced by cultured adipose-derived stem cells. These active materials, in relative concentrations secreted by the cultured cells, may be harvested and mixed with carefully selected proportions of transport ingredients. These transport ingredients aid in the delivery of the active materials to the living layers of the dermis.

### HADSCC Medium

This invention includes formulations containing human adipose-derived stem cell conditioned (HADSCC) media. Human stem cells, such as adipose-derived stem cells, produce a variety of growth-promoting and healing materials such as growth factors and cytokines (also referred as secretomes). While many of these have been identified, the cells likely also secrete other substances due to their pluri-potency either not yet known or with beneficial functions yet to be precisely identified. Some of these materials may be effective at low concentration. In some embodiments, the compositions of our invention include mixtures of growth factors and cytokines (including TGF-B, PDGF, and GM-CSG, interleukins, and matrix proteins) produced/secreted by cultured adipose-derived stem cells. These mixtures of stem cell products may be harvested from cultured adipose-derived stem cells by collecting the culture medium to which such cells have been exposed.

Adipose-derived stem cells are stem cells extracted from adipose tissues. Adipose tissue, like other tissue types, is not a homogenous mixture of a single cell type. Instead adipose tissue includes a combination of fat cells, vasculature, connective tissue, and blood cells. Human adipose tissue is available ex vivo as a result of various cosmetic procedures including liposuction. Stem cells may be extracted from such tissue by any of a number of methods known in the art, including treatment with surfactants or enzymes (including proteases such as such as collagenase, or trypsin), maceration, separation by centrifugation, filtering, or settling, ultrasonic treatment, adherent culturing, or some combination of these methods. Stem cells may also be extracted by the method disclosed in US pending application to Dhar et al. entitled Isolating adult stem cells from lipoaspirates and filed the same date as this PCT application

Once extracted from adipose tissue, adipose-derived stem cells may be grown in tissue by a number of methods known in the art, including growth on three-dimensional scaffolds or supports, growth in suspension culture, or growth on the surface of plastic or glass vessels.

Growth of such adipose-derived stem cells includes supply of nutrients for the cells through provision of an aqueous culture medium. Cells grow in culture in contact with medium and extract nutrients from it. These cells also deliver to the medium products of their growth and metabolism. Among the products are the growth factors and cytokines discussed above as well as metabolic products. Conventional tissue culture requires replacement of culture medium as cells use up the nutrients and deliver products that may affect future cell growth. This replacement may be either continuous, with a portion of the medium removed as new medium is added, or intermittent with periodic replacement of some or all of the culture medium in a vessel. Culture medium removed after exposure to cells in culture is known as spent or conditioned medium.

Cell culture also involves periodic “passaging” of cells. Passaging is formation of a subculture from an established cell culture. The purpose of passaging is to maintain cells in a growing state. Once cells grown in adherent culture reach confluence, so that the entire accessible surface of the culture vessel is covered with cells, the cells cease most division and growth. At confluence, the level of production of stem cell products may decline. In essence, the cells no longer need to communicate the message to grow further and exhibit a contact inhibition property. This is an important control to prevent unbridled proliferation en vivo, but it does not support maximal production of stem cell products in vitro. Once cells reach or approach confluence, some of the cells may be passaged by transferring them to a fresh culture vessel so that growth phase (and attendant production of the optimal mix of stem cell products) continues. Cells may be passaged by any of a number of techniques known in the art such as treatment by proteases that reduce adherence of the cells to the culture vessel; by simple washing with agitation that randomly releases some number of attached cells or by scrapping off with a sterile cell scrappers. Once released, the cells may be transferred to a fresh vessel (or vessels) with fresh culture medium. These released cells may then resume growth by attaching to a surface of the fresh vessel. We have found that our adipose-derived stem cells produce maximally effective concentrations of stem cell products when the cells are grown at less than full confluence, and preferably at about 40% to about 90% confluence. In some embodiments, adipose-derived stem cells produce maximally effective concentrations of stem cell products when the cells are grown at about 70% confluence. Maintenance of stem cells at this level of confluence has the added benefit of at least partially synchronizing the cell cycle phase of the growing cells so that the stem cell products produced reflect the activity of growing cells.

Culture medium compositions typically include essential amino acids, salts, vitamins, minerals, trace metals, sugars, lipids, and nucleosides. Cell culture medium attempts to supply the components necessary to meet the nutritional needs required to grow cells in a controlled, artificial and in vitro environment. Nutrient formulations, pH, and osmolarity may vary depending on the type of cell cultured, on cell density, and on the culture system employed. The scientific literature includes description of many cell culture medium formulations; a number of such media are commercially available. Conditioned medium contains many of the original components of the medium, as well as a variety of cellular metabolites and secreted proteins, including, for example, biologically active growth factors, inflammatory mediators and other extracellular proteins. Examples of suitable culture media are Dulbecco's Modified Eagle's Medium and RPMI 1640. Such media may be supplemented by other nutrients or growth supporting materials as is well known in the art.

The mix and concentration of stem cell products depends on many factors, such as the number of active cells per unit volume of culture medium, the time between changes of the medium (or effective turnover time when medium is changed on a continuous basis), the type of stem cell, the growth phase or mitotic phase of the cells, the density of cells on the support (if one is used), and many other factors. There is not a simple relationship between stem cell product concentration and time between medium changes because as this time increases, the cells exhaust nutrients from the medium so that the rate of production of stem cell products may decrease. Also, stems cells grow and divide in culture; more cells contribute to stem cell products production. Further, stem cell products themselves may serve a communication function among the cells, depressing or enhancing the production of certain stem cell products by cells, and directly or indirectly affecting the cell growth rate or mitotic stage. Indeed, we believe that the value in treating damaged or aging skin or other tissue arises because the stem cell products have effects on other cells.

A medium change means that effectively all of the medium in a culture vessel is removed by pipetting or decanting and fresh medium is added to the vessel. We have found that the highest production rate for stem cell products occurs when the time between medium changes is less than about once every 72 hours and preferably about once every 48 hours. We have further found that we can maximize the production rate of an effective mix of stem cell products by passaging cultures so as to maintain between about 40% to about 90% confluence with change of medium for product formulation less than about once every 60 hours. In some embodiments, we produce maximally effective stem products by changing medium twice about 48 hours apart while maintaining confluence at about 70%. Since the cells are continuously growing and thus increasing the degree of confluence between medium changes, maintaining confluence at about 70% means that we target a 70% confluence between two medium changes. That is, a first change occurs at lower than 70% confluence and a second change occurs at higher than 70% confluence, with 70% confluence occurring between the two changes. In some embodiments, the compositions of the invention include a mixture of material from two changes of medium removed from the same culture at two different times. This has the benefit of producing a more consistent product. In each case, the changes used in the mixture are drawn from cultures about 40% to about 90% confluence and may be chosen so that 70% confluence occurs between two changes used in the mixture.

The amount of media in each change depends on the size of the culture vessel. Conventional culture flasks have substantially flat bottoms and range in area from about 25 to about 225 cm². In embodiments where the cells are adherent to the walls of the flask, the amount of medium may be controlled by adjusting the depth of fill to about a constant value. For example, if each flask were filled to a depth of 0.5 cm, then each 1 cm² of growth area (and of cells at confluence) would correspond to a culture medium volume of 0.5 mL. We have found that fill depths of about 0.2 cm to about 1.0 cm produce useable concentrations of stem cell products, but that a depth of 0.15-0.25 cm (achieved by adding between 12 and 50 mL to flasks with respective area of 75-225 cm²) produces a particularly effective amount with a 48 hour medium change interval centered on 70% confluence.

An important characteristic of the formulations of the invention is that they do not include added materials that may further damage skin. Accordingly, in some embodiments, the formulations do not include any of: parabens, synthetic dyes, petrochemicals, phthalates, or triclosan. The formulations also do not include products of Genetically Modified Organisms.

Without intent to be bound by theory, one of the results of aging is that the communication process between skin cells, which is mediated by growth factors, is reduced. As a consequence, the activity of fibroblasts decreases. This may strongly affect the extracellular matrix (ECM), the structural network of the skin. The reduced synthesis of natural growth factors affects the structural network of the skin. The result of this is a reduction in the skin's firmness, elasticity, and density.

### Autologous Stem Cells

As discussed above, tissue-derived products, particularly those derived from minimally processed human tissue, may contain infectious agents or antigens. The invention includes compositions containing HADSCC media derived from stem cells autologous with the treated individual. Such compositions are not necessarily free of infectious agents or antigens, but the treated individual has already been exposed to these autologous agents and antigens so that the risk of further infection or immune reaction should be low. In some embodiments, the stem cell cultures that produce the HADSCC medium include stem cells selected by adherent culture of stromal vascular fraction cells derived from autologous lipoaspirates. Such lipoaspirates may be available from cosmetic procedures such as cosmetic liposuction.

These “autologous dermal products” are of particular benefit when the individual has compromised immune status or has an autoimmune condition with particular sensitivity to allogenous antigens.

The process for preparing autologous HADSCC media is the same as that described for non-autologous media, except that the cultures are specific for each individual. The dermal products including autologous HADSCC media are also specific for each individual. The invention includes a process of producing individual-specific dermal products according to predetermined recipes that depend on the targeted portion of the skin and on the targeted condition. The recipes are substantially the same as other dermal products described in this disclosure, except that the HADSCC media used is drawn from stem cell cultures derived from the treated individual. The process includes steps of identifying a specific dermal product requirement, preparing HADSCC media from autologous cells derived from a lipoaspirate from an individual, and formulating the dermal product according to the recipe.

The batch quantities may be much smaller for autologous dermal products so the formulation step is correspondingly scaled down. Where a standard dermal product might be produced on about 100 kg production-scale equipment such as temperature-controlled compounding vats, autologous dermal products may be produced in 10 g to 500 g boutique quantity, depending on the particular product. Typically, these materials are compounded in small scale labware.

### Transport Ingredients

Topically applied cosmetics are largely unable to reach dermal fibroblasts because of their depth within the skin and the intervening relatively impervious layers including the stratum corneum. Keratinocytes underlying the stratum corneum produce the corneocytes, but are themselves not easily reached. Without intent to be bound by theory, successful delivery of stem cell products to keratinocytes can indirectly affect deep fibroblasts as the keratinocytes communicate with the fibroblasts through the interstitial components of the deeper skin. Appropriate mix of stem cell products that reach keratinocytes may cause the keratinocytes to in turn produce growth factors that communicate with the deeper skin layers. We have invented formulations that deliver effective amounts of appropriate growth factors by combining a mixture of stem cell products harvested from adipose-derived stem cells according to our protocols with selected delivery agents. The selection and proportion of delivery agents serve to target different aspects of the skin and thus treat different dermal conditions by stimulating keratinocytes to secrete growth factors. These growth factors enhance the synthesis of collagen and elastin in the dermis.

The effect of this stimulation process is to stimulate natural growth factors of the skin and boost collagen and elastin production thereby renewing the skin's resilience and firmness.

Transport ingredients serve to allow the stem cell products to reach living layers of the skin in effective amounts. Because some transport routes differ, a higher concentration of stem cell products may be required to deliver an effective amount to some targets. The intended target of the product has a large effect on the difficulty of transport. For example, products that target hair follicles can follow the path of the hair shaft itself. The hair shaft penetrates the stratum corneum and provides a transport path directly to the follicle, which includes layers of living cells that produce the hair shaft. The hair shaft fills most of the opening through the stratum corneum, but the shaft may swell or shrink depending on materials applied, thereby opening up a relatively direct passage. In contrast, a foot cream must penetrate the stratum corneum on the plantar surface of the foot. Plantar skin is hairless and heavily keratinized. Thus transport along a hair shaft is not available. However plantar skin is densely covered with eccrine sweat glands. These sweat glands comprise secretory tubules connected through openings in the stratum corneum by excretory ducts. The excretory ducts are surrounded by a double layer of cuboidal epithelial cells. These ducts have cross-sections of 30 to 40 micrometers and reach deep into the dermal matrix. Other transport routes include through sebaceous glands proximal to follicles (particularly relevant for treating acne) and directly through the stratum corneum itself.

The stratum corneum resists aqueous transport both through its thickness and laterally. However, the underlying dermal tissue is much less resistant to lateral transport. Thus if therapeutic materials reach the underlying dermal tissue even in limited sites (such as at follicles, sweat glands, or sebaceous glands) lateral diffusion of these materials can eventually reach a larger portions of the dermis. Further, as discussed above, the action of growth factors that reach some cells may set up a cascade of growth factors produced by those cells that influence other cells.

Although the stratum corneum resists aqueous transport, it is much less resistant to transport by certain polar organic solvents such as DMSO. DMSO easily penetrates the skin and substances dissolved in the solvent may be quickly absorbed. DMSO has a toxicity profile inappropriate for dermal treatment, but its ability to penetrate the stratum corneum directly shows that this is not a completely impervious layer. Without intent to be bound by theory, we believe that certain of our formulations reach living layers of the skin at least in part through transport directly through the stratum corneum. The aqueous solutions of stem cell products and selected organic materials including oils and waxes combined with surface active materials including surfactants and emulsifiers produce emulsions that may have at least some ability to transport stem cell products through the stratum corneum.

The particular mix of transport ingredients depends upon the intended target and function of the formulation and upon the method of administration.

Methods of administration include “rinse off” formulations where the product is intended to be applied for a short time and then removed. Examples include shampoos, hair conditioners, cleansers, and washes. Non-rinse formulations are products applied and left in place for an extended time. Examples include skin sera, wrinkle treatments, lightening creams, foot creams, and acne treatments.

Transport ingredients work in tandem with HADSCC medium to control delivery because the HADSCC medium is an aqueous solution. Outer skin layers impede direct transport of aqueous materials, but the nature of the stem cell products requires that they remain in aqueous solution. Transport ingredients include organic phase components such as oils, plus surfactants and emulsifiers. A useful oil in this regard is coconut oil, which is a highly saturated fat with the demonstrated ability to form emulsions with (coconut) proteins. Since some stem cell products are proteins, coconut oil may be particularly suitable. Other organic phase components useful for as transport ingredients include argan oil, avocado oil, shea butter, white petrolatum, and caprylic/capric triglyceride, a trigyceride itself derived from coconut oil. Useful surfactants and emulsifiers include decyl glucoside, cocamidopropyl betaine, glycol stearate, polysorbate 20, lecithin, cetyl alcohol, and glycerol monostearate. Commercial blends of these materials (such as Jeesperse CPW-CGT brand of organic emulsion manufactured by Keen International of Fairfield NJ) may also be useful as transport ingredients when present in appropriate concentrations as detailed in the Examples.

### Support Ingredients

Some formulations of the invention include functions in addition to that of improving the condition of damaged skin. For example, a shampoo formulated to help regenerate hair should also clean hair. A hair conditioner with similar function should also moisturize and strengthen existing hair. A face cleanser should also clean and moisturize skin of the face. Support ingredients may address these more conventional functions. Such support ingredients may include: moisturizers (e.g. aloe vera gel), humectants (e.g. glycerol), nutrients (e.g. vitamins B3 and B5), and antioxidants (e.g. green tea extract). Other support ingredients serve ancillary functions related to shelf stability, product texture, and fragrance. For example, the formulations may include preservatives such as phenoxyethanol/sorbic acid/capryl glycol, natural antimicrobials such as grapefruit seed extract, fragrance, thickening agents such as methyl cellulose or cellulose gum, and pH adjusters such as citric acid.

We have selected effective combinations and proportions of HADSCC medium and transport ingredients as disclosed in further detail below. All percentage contents are by weight.

Non-rinse products targeted at distributed areas of the dermis, such as serum type products, lightening creams, and wrinkle creams products, are effective with high aqueous content (greater than about 75%) are combined with moderate surfactant/emulsifier concentration (about 5%) with minimal organic phase ingredients. The bulk of the aqueous content (about 55-80% of the total formulation by weight) may be HADSCC medium.

Non-rinse products directed to distributed areas of the dermis where the skin in heavily keratinized, such as foot cream directed to the plantar surface of the foot, are effective with much lower aqueous fractions. These may include moderate concentrations of HADSCC medium (about 40 to 60%), relatively high concentrations of organic phase materials (about 10-30%) and low concentrations of surfactants/emulsifiers (about 2-5%).

Certain rinse-off products targeted to hair follicles that also perform a cleaning function, such as shampoos, are effective with low amounts of HADSCC medium (about 10%), high amounts of surfactants/emulsifiers (about 70%) and relatively low organic phase ingredients (about 5%). This formulation is dominated by surfactants but the mode of application to wet hair increases the effective amount of aqueous phase materials.

Other rinse-off products targeted to hair follicles that do not perform a cleaning function, such hair conditioners, are effective with very high concentrations of HADSCC medium (about 75%) moderate concentrations of surfactants (about 5-10%) and relatively low organic phase ingredients (about 5%). Use of this product is intended to follow use of the shampoo described above and supplies a much higher concentration of stem cell products once materials that may block access to the follicles are washed away and transport pathways along hair shafts are made more pervious. One method of restoring and regenerating the condition of the hair is to first wash the hair using the surfactant-heavy formulation of shampoo, followed by more prolonged application of the stem-cell-product-rich conditioner.

### Excluded Ingredients

An important characteristic of the formulations of the invention is that they do not include added materials that may further damage skin. Accordingly, in some embodiments, the formulations do not include any of: parabens, synthetic dyes, petrochemicals, phthalates, or triclosan. The formulations also do not include products of Genetically Modified Organisms.

### Use of Products

The dermal product of the invention may be applied in a manner typical of conventional cosmetic and skin care products. No-rinse products are applied by rubbing the product into targeted areas of the skin. Rinse type products are removed after a period of application. The inventive hair care products are compounded to deliver an effective amount of stem cell products to hair follicles using different concentrations of media, transport, and support ingredients. A hair treatment method includes applying to the hair two described dermal treatment compositions. The first composition includes about 10% of the medium and about 70% of surfactants/emulsifiers by weight. Washing the hair with this first product has two results: first, the support ingredients clean the hair and scalp, removing dirt and oil surrounding the follicle. Second, the product may increase the permeability of the hair shaft within the follicle to subsequently applied materials. Without intent to be bound by theory, inventors believe that a follicle may either recede slightly from surrounding follicular tissue creating a larger channel, or may itself become more permeable through absorption of the transport materials. The second composition (applied as a conventional conditioner by massaging thoroughly into the wet hair until completely absorbed) includes about 5% coconut oil, about 75% of stem cell derived medium and about 5% to about 10% of surfactants/emulsifiers by weight. The high concentration of stem cell products allows a significant amount of these products to reach hair follicles. The transport ingredients (oil and surfactants) support both an aqueous entry point around the hair shaft as well as an organic phase transport of stem cell products. The method includes steps of washing the hair with the first composition, rinsing the hair with water to remove the first composition, applying to the hair the second composition, and rinsing the hair with water to remove the second composition. The second composition may remain in the hair for a longer time (typically from about one to about ten minutes) than the first composition (transiently, usually less than about one minute).

### EXAMPLES

We have combined these ingredients into formulations that deliver effective amounts of stem cell products to improve the appearance and condition of skin and hair that has been damaged or aged. The following non-limiting examples recite specific formulations. Other formulations are possible that may be evident to the skilled practitioner upon review of this document. The various formulations are characterized as rinse-off or non-rinse based in normal use. Some rinse-off compositions (e.g. face cleanse, shampoo) are rinsed off after transient exposure. Others (conditioner) may be left in place for several minutes. Non-rinse compositions are left in place to absorb into or otherwise transit to the target area of the skin.

Hair products (shampoo, conditioner, hair-restoring serum) are targeted to the hair follicles. Other products are applied to selected areas evident from the product name. Foot cream is applied to the feet. Eye serum to skin surrounding the eyes. Universal serum may be applied to any area of the skin.

The white and bright cream described may also contain other support materials as whitening agents such as vitamin B3 (niacinamide), hydroquinonne, and kojic acid to reduce the appearance of skin discolorations.

The listed ingredients (with weighed quantities adjusted for batch size) are typically combined in a cleaned and sanitized tank, with moderate mixing for standardized products. Autologous products are made in much smaller batches in disposable labware. The ingredients may be added in the order listed, one at a time, mixing must be well between additions. The final pH of the mixture may be adjusted if outside of the indicated range.

### Example 1

### Hair Regenerating Shampoo (Rinse-Off)

| Ingredient (pH range: 6.4-6.8) | Wt. Limit (%) |
|---|---|
| Cocamidopropyl Betaine | 20%-40% |
| Decyl Glucoside | 20-40% |
| HADSCC Medium | 5%-10% |
| Glycol Stearate/Stearamide AMP | 5%-10% |
| Coconut Oil | 5%-10% |
| Glycerin | 2%-5% |
| PEG-150 Distearate | 2%-5% |
| Grapefruit Seed Extract | 2%-5% |
| Aloe Vera Gel | 2%-5% |
| Phenoxyethanol/Sorbic acid /Caprylyl Glycol | 1%-3% |
| Propyltrimonium | 1%-3% |
| Fragrance | 1%-3% |
| Hydrolyzed Biotein Silk | 0.5%-1% |
| Cellulose Gum | 0.1%-0.5% |
| Hydrolyzed Wheat Protein | 0.1%-0.5% |
| Soy Rice Peptide | 0.1%-0.5% |
| Bamboo Extract | 0.1%-0.5% |
| Licorice Extract | 0.1%-0.5% |
| Argan Oil | 0.1%-0.5% |
| Avocado Oil | 0.1%-0.5% |
| Nanosome Copper Peptides | 0.001%-0.01% |
| EGF-1 (Human Oligopeptide-1) | 0.001%-0.01% |

### Example 2

### Hair Regenerating Conditioner (Rinse-Off)

| Ingredient (pH range = 6.40-6.80) | Wt. Limit (%) |
|---|---|
| HADSCC Media | 50%-90-% |
| Jeesperse CPW-CGT (Cetyl Alcohol/Sodium | 5%-10% |
| Acrylate/Sodium Acryloyl Dimethyl Taurate | |
| Copolymer/Glyceryl | |
| Monostearate/Caprylic/Capric Triglyceride) | |
| Coconut Oil | 5%-10% |
| Glycerin | 2%-5% |
| Aloe Vera Gel | 2%-5% |
| Grapefruit Seed Extract | 2%-5% |
| Propyltrimonium | 2%-5% |
| Phenoxyethanol/Sorbic acid/Caprylyl Glycol | 1%-3% |
| Fragrance | 1%-3% |
| DL-Panthenol (Vitamin B5) | 0.1%-0.5% |
| Hydrolyzed Wheat Protein | 0.1%-0.5% |
| Hydrolyzed Silk Biotein | 0.1%-0.5% |
| Bamboo Extract | 0.1%-0.5% |
| Licorice Extract | 0.1%-0.5% |
| Argan Oil | 0.1%-0.5% |
| Avocado Oil | 0.1%-0.5% |
| Nanosome Copper Peptides | 0.001%-0.01% |
| EGF-1(Human Oligopeptide-1) | 0.001%-0.01% |

### Example 3

### Universal Serum (Non-Rinse)

| Ingredient (pH range = 6.00-6.80) | Wt. Limit (%) |
|---|---|
| HADSCC Medium | 50-70.% |
| Palmitoyl tripeptide-5/Glycerin | 5-10.0% |
| Acetyl Hexapeptide-8 (Argireline) | 5-10.0% |
| Polysosbate-20 | 5-10.0% |
| Acetyl Tetrapeptide-5 (Eyeseryl) | 5-10.0% |
| Aloe Vera Gel | 5-10.0% |
| Pentapeptide-18/Caprylyl Glycol (Leuphasyl) | 3-7.0% |
| Algae Extract/Pullulan | 1-5.0% |
| Grapefruit Seed Extract | 1-4.0% |
| Sodium Carboxymethyl Cellulose | 1-3.0% |
| Phenoxyethanol/Sorbic acid/Caprylyl Glycol | 1-1.5% |
| Sodium Hyaluronate Solution | 1-3.0% |
| Fragrance | 1-3.0% |
| DL-Panthenol (Vitamin B5) | 1-3.0% |
| Niacinamide (Vitamin B3) | 1-3.0% |
| Green Tea Extract | 1-3.0% |
| Nanosome Copper Peptides | 0.001-0.01% |
| EGF-1(Human Oligopeptide-1) | 0.001-0.01% |

### Example 4

### Face Wash (Rinse-Off)

| Ingredient (pH range = 6.00-6.80) | Wt. Limit (%) |
|---|---|
| HADSCC Medium | 10-30% |
| Decyl Glucoside | 10-40.0% |
| Cocamidopropyl Betaine | 10-30.0% |
| Glycerin | 5-10.0% |
| Polysorbate-20 | 5-10.0% |
| Aloe Vera Gel | 5-10.0% |
| Phenoxyethanol/Sorbic acid/Caprylyl Glycol | 1-1.5% |
| Citric Acid-20% | 1-3.0% |
| Fragrance | 1-3.0% |
| Cellulose Gum | 1-3.0% |
| Quillaja Saponia | 1-3.0% |
| Sodium PCA | 1-3.0% |
| Grapefruit Seed Extract | 1-3.0% |
| Green Tea Extract | 1-3.0% |
| Calendula Extract | 1-3.0% |
| Nanosome Copper Peptides | 0.001-0.01% |
| EGF-1(Human Oligopeptide-1) | 0.001-0.01% |
| FD & C Red 40 (1% Solution) | 0.001-0.01% |

### Example 5

### Anti Hair Loss Serum (Non-Rinse)

| Ingredient (pH range = 5.50-5.90) | Wt. Limit (%) |
|---|---|
| Propylene Glycol | 50%-65% |
| HADSCC Medium | 20%-40% |
| Polysobate-20 | 5%-10% |
| Minoxidil Sulfate | 2%-10% |
| Aloe Vera Gel | 1%-3% |
| Hydrolyzed Keratin | 1%-3% |
| Hydrolyzed Wheat Protein | 1%-3% |
| Propyltrimonium | 1%-3% |
| Grapefruit Seed Extract | 1%-3% |
| Chamomile Extract | 1%-3% |
| Willow Bark Extract | 1%-3% |
| Green Tea Extract | 1%-3% |
| Rosemary Extract | 1%-3% |
| Capixyl | 1%-3% |
| Phenoxyethanol/Sorbic Acid/Caprylyl Glycol | 1%-3% |
| Fragrance | 1%-3% |
| Sodium Hydroxide | 0.1%-0.5% |
| Vitamin B 3, 5, 6 | 0.1%-0.5% |
| Cellulose Gum | 0.1%-0.5% |
| Sodium PCS | 0.1%-0.5% |
| Zinc Pyrithione | 0.01-0.1 |
| Allantoin | 0.01-0.1 |
| Nanosome Copper Peptides | 0.001-0.01 |

### Example 6

### Universal Cream (Non-Rinse)

| Ingredient (pH range = 6.40-6.80) | Wt. Limit (%) |
|---|---|
| HADSCC Medium | 50-70.% |
| Jeesperse CPW-CGT (Cetyl Alcohol/Sodium | 5-10.0% |
| Acrylate/Sodium Acryloyl Dimethyl Taurate | |
| Copolymer/Glyceryl | |
| Monostearate/Caprylic/Capric Triglyceride) | |
| Agireline-NP (Acetyl Hexapeptide-8) | 5-10.0% |
| Eyeseryl | 5-10.0% |
| Pentapeptide-18/Capryly1Glycol | 3-7.0% |
| Eyeliss (hesperidin methyl chalcone, valyl- | 2-5.0% |
| tryptophane, and lipopeptide PAL-GQPR) | |
| Argan Oil | 1-4.0% |
| Haloxyl | 1-4.0% |
| Aloe Vera Gel | 1-4.0% |
| Shae Butter | 1-4.0% |
| Vitamin E (DL-Alpha Tocopheryl Acetate) | 1-4.0% |
| Vegetable Squalane | 1-4.0% |
| Fragrance | 1-4.0% |
| Phenoxyethanol-SA | 1-1.5% |
| Dimethicone | 1-3.0% |
| Vitamin B 3 (Niacinamide) | 1-3.0% |
| Vitamin C Oily (Tetrahexyldecyl Ascorbate) | 1-3.0% |
| Grapefruit Seed Extract | 1-3.0% |
| Green tea Extract | 1-3.0% |
| Licorice Extract | 1-3.0% |
| Lecithin | 1-3.0% |
| Sodium Hyaluronate | 1-3.0% |
| Co-Enzyme Q-10 | 1-3.0% |
| Alpha Arbutin | 1-3.0% |
| Vitamin B 5 (DL-Panthenol) | 1-3.0% |
| Allantoin | 0.01-1.0% |
| Soy Rice Peptide | 0.01-1% |
| Copper Peptides | 0.001-0.01% |
| EGF-1/CG-EGF | 0.001-0.01% |
| EGF-2/CG-IGF-1 | 0.001-0.01% |
| EGF-3/CG-bFGF | 0.001-0.01% |
| EGF-4/CG-TRX | 0.001-0.01% |

### Example 7

### Eye Serum (Non-Rinse)

| Ingredient (pH range = 6.40-6.80) | Wt. Limit (%) |
|---|---|
| HADSCC Medium | 50-70.% |
| Acetyl Hexapeptide-8 (Argireline) | 5-10.0% |
| Niacinamide (Vitamin B3) | 5-10.0% |
| Polysosbate-20 | 5-10.0% |
| Acetyl Tetrapeptide-5 | 5-10.0% |
| Aloe Vera Gel | 5-10.0% |
| Pentapeptide-18/Caprylyl Glycol (Leuphasyl) | 3-7.0% |
| Grapefruit Seed Extract | 1-4.0% |
| Sodium Carboxymethyl Cellulose | 1-3.0% |
| Phenoxyethanol/Sorbic acid/Caprylyl Glycol | 1-1.5% |
| Sodium Hyaluronate Solution | 1-3.0% |
| Fragrance | 1-3.0% |
| Alpha Arbutin | 1-3.0% |
| DL-Panthenol (Vitamin B3) | 1-3.0% |
| Green Tea Extract | 1-3.0% |
| Nanosome Copper Peptides | 0.001-0.01% |
| EGF-1(Human Oligopeptide-1) | 0.001-0.01% |
| FD & C Red 40 (1% Solution) | 0.001-0.01% |

### Example 8

### Foot Cream (Non-Rinse)

| Ingredient | Limit (%) |
|---|---|
| HADSCC Medium | 40%-70% |
| Jeesperse CPW-CG-T (Cetyl Alcohol/Sodium | 5%-10% |
| Acrylate/Sodium Acryloyl Dimethyl Taurate | |
| Copolymer/Glyceryl | |
| Monostearate/Caprylic/Capric Triglyceride) | |
| White Petrolatum | 5%-10% |
| Aloe Vera Gel | 1%-5% |
| Grapefruit Seed Extract | 1%-5% |
| Glycerin | 1%-5% |
| Shea butter | 1%-5% |
| Coconut Oil | 1%-5% |
| Safflower Oil | 1%-5% |
| Vegetable Squalane (Olive) Oil | 1%-5% |
| Vitamin E (DL-Alpha Tocopheryl Acetate) | 1%-5% |
| Rosemary Extract | 0.5%-1% |
| Phenoxyethanol/Sorbic acid/Caprylyl Glycol | 0.5%-1% |
| Fragrance | 0.5%-1% |
| DL-Panthenol (Vitamin B-5) | 0.5%-1% |
| Honey | 0.5%-1% |
| Sea Kept Extract | 0.5%-1% |
| Calendula Extract | 0.1%-0.5% |
| Rosehip Extract | 0.1%-0.5% |
| Vitamin A (Retinol) | 0.1%-0.5% |
| Vitamin C Oily (Tetrahexyldecyl Ascorbate) | 0.1%-0.5% |
| Tea Tree Oil | 0.1%-0.5% |
| Argan Oil | 0.1%-0.5% |
| Eucalyptus Oil | 0.01%-0.1% |
| Peppermint Oil | 0.01%-0.1% |
| Menthol | 0.01%-0.1% |
| Nanosome Copper Peptides | 0.001%-0.01% |
| EGF-1(Human Oligopeptide-1) | 0.001%-0.01% |

### Example 9

### Acne Cream (Non-Rinse)

| Ingredient | Wt. Limit (%) |
|---|---|
| HADSCC Medium | 45%-75% |
| Jeesperse CPW-CG-T (Cetyl Alcohol/Sodium | 10%-14% |
| Acrylate/Sodium Acryloyl Dimethyl Taurate | |
| Copolymer/Glyceryl | |
| Monostearate/Caprylic/Capric Triglyceride) | |
| Butylene Glycol | 5%-10% |
| Vitamin B3 (Niacinamide) | 5%-10% |
| Glycolic Acid 70% (Active Ingredient) | 5%-10% |
| ZincOxide/Triethoxycaprylylsilane/C12-15Alkyl | 2%-5% |
| Benzoate/DEA Oleth3 Phosphate | |
| Methylsulfonylmethane (MSM) | 2%-5% |
| Vitamin C Oily (Tetrahexyldecyl Ascorbate) | 1%-4% |
| Borage Water (1%) | (1%-3%) |
| Salicylic Acid | (1%-3%) |
| Willow Bark Extract | (1%-3%) |
| Multivitamin (A, F, E, B-5 . . . Lecithin) | (1%-3%) |
| Witch Hazel | (1%-3%) |
| Honey | (1%-3%) |
| Phenoxyethanol/Sorbic acid /Caprylyl Glycol | (1%-3%) |
| Tetrapeptide-14 | (1%-3%) |
| Grapefruit Seed Extract | (1%-3%) |
| Tea Tree Oil | (0.1%-1.5%) |
| Maltodextrin/Syringa Vulgaris (Lilac) Leaf Cell | (0.1%-1.5%) |
| Culture) | |
| Green Tea Extract | (0.1%-1.5%) |
| Fragrance | (0.1%-1.5%) |
| Calendula Extract | (0.1%-1.5%) |
| Cellulose Gum | (0.1%-1.5%) |
| Rosehip Oil | (0.1%-1.5%) |
| Idebenone | (0.01%-0.1%) |
| Allantoin | (0.01%-0.1%) |
| Nanosome Copper Peptides | (0.001%-0.01%) |
| EGF-1 (Human Oligopeptide-1) | (0.001%-0.01%) |

### Example 10

### Light and Bright Cream (Non-Rinse)

| Ingredient | Wt. Limit (%) |
|---|---|
| HADSCC Media | 57% |
| Transport Ingredients: | 8% |
| Jeesperse CPW-CG-T (Cetyl Alcohol/Sodium | |
| Acrylate/Sodium Acryloyl Dimethyl Taurate | |
| Copolymer/Glyceryl Monostearate/Caprylic/Capric | |
| Triglyceride) | |
| Argan Oil | 1% |
| Support Ingredients | 34% |

### Example 11

### Universal Serum II (Non-Rinse)

| Ingredient | Wt. Limit (%) |
|---|---|
| HADSCC Media | 70.00% |
| Palmitoyl tripeptide-5/Glycerin | 5.00% |
| Acetyl Hexapeptide-8 (Argireline) | 5.00% |
| Polysosbate-20 | 5.00% |
| Acetyl Tetrapeptide-5 | 3.00% |
| Aloe Vera Gel | 2.00% |
| Pentapeptide-18/Caprylyl Glycol(Leuphasyl) | 2.90% |
| Algae Extract/Pullulan | 1.00% |
| Grapefruit Seed Extract | 1.00% |
| Sodium Hydroxymethlyl Cellulose | 1.00% |
| Phenoxyethanol/Sorbic acid/Caprylyl Glycol | 1.00% |

### Example 12

### Advance Infusion Serum (Non-Rinse)

| Ingredient | Wt. (%) |
|---|---|
| HADSCC Media | (10%-30%) |
| Deionized Water | (50%-60%) |
| Hyaluronic Acid 0.5% Solution | (3%-10)% |
| Aloe Vera Gel | (3%-10)% |
| Grapefruit Seed Extract | (3%-5%) |
| Citric Acid 20% Solution | (0.2%-1%) |
| 1,2 Hexanediol, Caprylyl Glycol, Troplone | (0.2%-1%) |
| Vitamin A, F, E, B3, B5, Lecithin | (0.001%-0.01%) |
| Nanosome Palmitoyl Pentapeptide-3 | (0.001%-0.01%) |
| Acetyl Glutamyl Heptapeptide-1 | (0.001%-0.01%) |
| Nanosome Copper Peptide | (0.001%-0.01%) |
| Green Tea Extract | (0.001%-0.1%) |

### Example 12

### Toner (Non-Rinse)

| Ingredient | Wt. (%) |
|---|---|
| HADSCC Media | 50%-80% |
| Butylene Glycol | 5%-10% |
| Aloe Vera Gel | 5%-10% |
| Polysorbate-20 | 5%-10% |
| Glycolic Acid 70% | 1%-5% |
| Witch Hazel | 1%-5% |
| Rosemary Extract | 1%-5% |
| Grapefruit Seed Extract | 1%-5% |
| Phenoxyethanol/Sorbic acid/Caprylyl Glycol | 1%-5% |
| Sodium Hyaluronate Solution | 1%-5% |
| Chamomile Extract | 0.1%-1% |
| Willow bark Extract | 0.1%-1% |
| Green Tea Extract | 0.1%-1% |
| White Tea Natural Fragrance | 0.1%-1% |
| Sodium PCA | 0.1%-1% |
| Salicylic Acid | 0.1%-1% |
| Vitamin B3 ( Niacinamide) | 0.1%-1% |
| Vitamin B5 (DL-Panthenol) | 0.1%-1% |
| Allantoin | 0.01%-0.1% |
| Menthol | 0.001%-0.01% |
| Nanosome Copper Peptides | 0.001%-0.01% |

Materials identified as Jeesperse are marketed by Jeen International Corp of Fairfield, N.J.

This specification discloses various aspects of the invention with reference to particular embodiments, but it should be understood that any of the features, functions, materials, or characteristics may be combined with any other of the described features, functions, materials, or characteristics. The description of particular features, functions, materials, or characteristics in connection with a particular embodiment is exemplary only; it should be understood that it is within the knowledge of one skilled in the art to include such feature, structure, or characteristic in connection with other embodiments whether or not explicitly described. We intend the scope of the appended claims to encompass such alternative embodiments. Variations on these described embodiments will become apparent to those of ordinary skill in the art upon reading the description. The inventors expect skilled artisans to employ such variations as appropriate, and the inventors intend for the invention to be practiced otherwise than specifically described herein. Accordingly, this specification and claims include all modifications and equivalents of the subject matter recited in the claims appended hereto as permitted by applicable law.

Unless otherwise indicated, all numbers used in the specification and claims are to be understood as being modified in all instances by the term “about.” Unless indicated to the contrary, the numerical values in the specification and attached claims are approximations that may vary depending upon the desired properties sought to be obtained.

The terms “a,” “an,” “the” and similar referents used in the context of describing the invention (especially in the context of the following claims) are intended to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the claims. No language in the specification should be construed as indicating any non-claimed element essential to the practice of the invention.

1. A dermal treatment composition comprising a medium recovered from an adipose-derived stem cell culture wherein the culture is an adherent culture having from about 40% to about 90% confluence.

2. The dermal treatment composition of claim 1 wherein the medium includes a mixture of two changes of media removed less than about 60 hours apart.

3. The dermal treatment composition of claim 2 wherein the culture reaches a confluence of about 70% between the two changes of media, wherein the time between the two changes of media is about 48 hours, and wherein the culture is maintained in a flat-bottom vessel having a fill level of medium is about 0.15 to 0.25 cm.

4. The dermal treatment composition of claim 1, wherein the composition is a non-rinse composition including greater than about 75% of an aqueous content and about 5% of a surfactant/emulsifier by weight.

5. The dermal treatment composition of claim 4 wherein the composition includes about 70% of the medium by weight.

6. The dermal treatment composition of claim 1, wherein the composition is a non-rinse composition including about 40% to about 60% of the medium, about 10% to about 30% of organic phase materials, and about 2% to about 5% of surfactants/emulsifiers by weight.

7. The dermal treatment composition of claim 6 where the composition is a foot cream.

8. The dermal treatment composition of claim 1 wherein the composition is a rinse-off composition including about 10% of the medium and about 70% of surfactants/emulsifiers by weight.

9. The dermal treatment composition of claim 8 wherein the composition is targeted to hair follicles and wherein the composition further includes about 5% of organic phase ingredients by weight.

10. The dermal treatment composition of claim 3 wherein the composition is a non-rinse composition targeted to hair follicles and includes about 75% of the medium, about 5% of organic phase ingredients, and about 5% to about 10% of surfactants/emulsifiers by weight.

11. The dermal treatment composition of claim 3 wherein the composition includes a whitening agent.

12. The dermal treatment composition of claim 3 wherein the composition comprises a fat.

13. The dermal treatment composition of claim 13 wherein the fat includes one or more of coconut oil, avocado oil, or caprylic/capric triglyceride.

14. A method of treating hair comprising:
a. washing the hair with the composition of claim 3 wherein the composition includes about 10% of the medium and about 70% of surfactants/emulsifiers by weight;
b. rinsing the hair with water to remove the composition;
c. applying to the hair a mixture including about 5% coconut oil, about 75% of a second medium recovered from an adipose-derived stem cell culture, and about 5% to about 10% of surfactants/emulsifiers by weight;
d. rinsing the hair with water to remove the mixture,
wherein the mixture remains in the hair for a time longer than the composition.

15. An autologous dermal treatment composition for application to the skin of an individual, the composition comprising:
a. a medium recovered from an adipose-derived stem cell culture, wherein the stem cell culture is derived from stem cells of the individual;
b. wherein the culture is an adherent culture having from about 40% to about 90% confluence. The dermal treatment composition of claim 1 wherein the medium includes a mixture of two changes of media removed less than about 60 hours apart.

16. The composition of claim 15 including about 70% of the medium by weight.

17. The composition of claim 15 further comprising a whitening agent.

18. The composition of claim 15, further comprising:
c. about 5% of organic phase ingredients.

19. The composition of claim 18, further comprising about 5% to about 10% of surfactants/emulsifiers by weight.

20. The composition of claim 18, wherein the organic phase ingredients includes one or more of coconut oil, avocado oil, or caprylic/capric triglyceride.
